(19) United States
(12) Patent Application Publication
(10) Pub. No.: US 2003/0087278 A1
(43) Pub. Date: May 8, 2003

(54) Nuclease assay

(76) Inventors: Jon Sayers, Sheffield (GB); Min Feng, Austin, TX (US)

Correspondence Address: John R. Van Amsterdam, Wolf, Greenfield & Sacks, P.C., Federal Reserve Plaza, 600 Atlantic Avenue, Boston, MA, 02210, US

(21) Appl. No.: 10/218,700

(22) Filed: Aug. 14, 2002

Related U.S. Application Data
(60) Provisional application No. 60/312,736, filed on Aug. 15, 2001.

(51) Int. Cl. C12Q 1/68; C12P 19/34

(52) U.S. Cl. 435/6; 435/91.2

(57) ABSTRACT

Methods are provided which utilize the endonuclease activity of exonucleases wherein reaction conditions are provided which suppress detectable exonuclease activity but which retain or enhance the endonuclease activity of said exonuclease.

### RELATED APPLICATION

This application claims the benefit under 35 U.S.C. §119(e) of U.S. provisional application No. 60/312,736, filed Aug. 15, 2001, the entire disclosure of which is incorporated herein by reference.

### FIELD OF THE INVENTION

The invention relates to a method which utilizes the endonuclease activity of exonucleases characterized in that reaction conditions are provided which suppress detectable exonuclease activity but which retain or enhance the endonuclease activity of said exonuclease.

### BACKGROUND OF THE INVENTION

DNA metabolism involves a number of distinct enzyme activities involved in a variety of processes related to the synthesis, degradation and function of DNA. For example, and not by way of limitation, these include DNA replication; DNA recombination and repair; regulation of gene expression; stabilization of chromosomes; and the segregation of chromosomes during mitosis and/or meiosis. A vast array of enzyme activities are involved in regulating these processes.

Many DNA polymerases, apart from their DNA synthesizing properties, have exonuclease activities. These activities digest DNA either in a 3′→5′ direction or in a 5′→3′ direction. For example, and not by way of limitation, the 5′→3′ exonuclease domain of E. coli DNA polymerase I (ECPolI) has major roles in replication, DNA repair and recombination, including processing the Okazaki fragments formed on the lagging strand during DNA synthesis. Similar enzyme activity must exist in all cells, if only to process Okazaki fragments.

In addition, a number of 5′→3′ exonuclease enzymes have been identified which are not DNA polymerases but separate enzymes. Genes encoding many prokaryotic 5′→3′ exonucleases have been identified and they show a number of highly similar sequence elements between each other and with respect to DNA polymerases. This implies a conserved biochemical mechanism of action.

Recently, a number of eukaryotic 5′→3′ exonucleases have been purified and their sequences were shown to be similar to their prokaryotic counterparts (Leiber et al (1997) Bioessays 19, 233-40 ). Moreover, given the conservation in structural features of these enzymes it is not surprising that mutations in genes encoding 5′→3′ exonucleases have deleterious effects on cells carrying these mutations.

This large family of enzymes possess 5′→3′ exonuclease activity on duplex DNA with a free 5′-terminus, such as blunt-ended duplexes and on oligonucleotides annealed to a complimentary template. In addition, circular duplex DNA molecules containing a nick are also substrates for exonuclease activity and are converted to partially gapped or fully singled-stranded circular products.

In addition to the 5′→3′ exonuclease activity, many of these enzymes also display structure-specific endonuclease activity. Bifurcated structures are cleaved at or close to the site of branching by the structure-specific endonuclease component of the 5′→3′ exonuclease (see FIG. 1). Examples of 5′→3′ exonucleases containing endonuclease activity, include, amongst others, T7 gene 6 exonuclease, and the DNA Pol I enzymes from Escherichia coli and Thermus aquaticus, which show structure specific DNA binding and endonucleolytic cleavage of certain substrates. Moreover, the phage exonuclease T5 D15 exonuclease is an example of a single stranded endonuclease which can also process circular DNA molecules.

All of the above described exonuclease and endonuclease activities have been exploited to provide sensitive DNA genotyping assays referred to as Cleavase® and Taqman®.

The Cleavase® assay exploits both the exonuclease and endonuclease activities of Thermus aquaticus DNA polymerase I. The Cleavase® assay is a means to rapidly identify known and unknown mutations at specific gene loci.

Briefly, target DNA molecules are heat denatured to separate double stranded DNA and prevent formation of any higher order structures. The strands are rapidly cooled to prevent reannealing of complementary strands. Once cooled, secondary structures form in the separated DNA strands which are dependant on the primary DNA sequence. Typically, these are stem-loop structures which are substrates for the exonuclease and endonuclease activities of T. aquaticus DNA polymerase I. Single base changes in the primary DNA sequence can alter the secondary structures formed after separation and cooling. These altered secondary structures are also substrates for endonuclease digestion resulting in differential patterns of labelled DNA fragments when the assay products are separated and compared to the wild-type or control DNA.

Upon termination of the nuclease reaction the digestion products are separated by conventional denaturing polyacrylamide gel electrophoresis. The strands are labelled by conventional means prior to digestion (e.g. radio-isotopic, fluorescence) and visualized by autoradiography to produce a “bar code” typical of a specific DNA. Variations in the pattern of DNA fragments between wild-type and test DNA reveals a polymorphism typical of a mutation within the test DNA. The Cleavase® assay is described in U.S. Pat. No. 5,719,028, which is incorporated by reference.

An alternative genotyping assay is described by the so called “Taqman®” assay. This PCR based assay exploits the endonuclease activity of thermostable DNA polymerase and a single fluorescently labelled oligonucleotide which has been modified by the provision of two fluorescent tags, one positioned at the 5′ terminus of the oligonucleotide, the other at the 3′ terminus. The tagged oligonucleotide (“reporter”) is designed to anneal to a site in a gene potentially containing a mutation of interest. The annealed reporter is flanked by two additional oligonucleotides designed to anneal up-stream and down-stream of the reporter.

If the test DNA lacks the mutation of interest, the tagged oligonucleotide anneals entirely with the target sequence leaving no free 5′ end or region of non-complementarity between the target and reporter. The polymerase used in the PCR reaction polymerizes between the two flanking oligonucleotides incorporating the reporter into the PCR product. To detect the incorporation of the reporter into the PCR product, the PCR reaction is exposed to UV excitation which results in fluorescent resonance energy transfer (FRET) between the 5′ and 3′ fluorophores on the reporter. If there is mis-matching between the reporter and the target DNA the endonuclease activity removes the 5′ terminus of the reporter thus removing the 5′ fluorophore. This results in an unstable hybrid between the reporter and target DNA and failure to incorporate the reporter into the amplified PCR product. Upon excitation with UV there is no detectable signal which is indicative of the presence of a mutation in the DNA. The Taqman® assay provides a rapid mass screening method for the identification of mutations.

There are some 4000 genetic disorders which afflict mankind or predispose him to disease. Clearly means to rapidly and accurately identify genetic polymorphisms is highly desirable since this will allow clinicians to diagnose a particular disease and offer either therapy or prophylactic treatment.

In each of the above assays the 5′→3′ exonuclease used contains both exonuclease and endonuclease activities. The endonuclease activity is relatively easy to control since there are a finite number of secondary structures that can form with a primary DNA sequence under a set of defined, controllable conditions. However exonuclease activity is processive and in the presence of a free 5′ end the exonuclease will digest the substrate to completion if the assay is not terminated.

In PCT application WO01/09343, which is incorporated by reference in its entirety, a solution to this problem is provided. WO01/09343 discloses mutations in wild-type exonucleases which lack detectable exonuclease activity but retain endonuclease activity and a wild-type binding constant for their DNA substrates.

### SUMMARY OF THE INVENTION

We herein disclose an alternative approach to providing a solution to the problem of suppressing the exonuclease activity of unmodified exonucleases which does not affect the endonuclease activity of said exonuclease.

The present invention uses a reduction in the concentration of a metal ion co-factor to selectively suppress the exonuclease activity of an exonuclease having both exonuclease and endonuclease activity. The invention thus provides a method for suppressing the exonuclease activity of an exonuclease having both exonuclease and endonuclease activity, comprising contacting the exonuclease with a metal ion co-factor at a concentration sufficient to suppress the exonuclease activity without suppressing its endonuclease activity to such an extent.

The invention includes a method for exposing a nucleic acid to the endonuclease activity of an exonuclease having both exonuclease and endonuclease activity, comprising forming a preparation containing the nucleic acid, the exonuclease and a metal ion co-factor at a concentration sufficient to suppress the exonuclease activity without suppressing its endonuclease activity to such an extent.

According an aspect of the invention there is provided an assay for comparing a nucleic acid with a comparison nucleic acid sequence comprising:

i) providing a sample of nucleic acid to be assayed;

ii) providing conditions for the denaturation of the nucleic acid;

iii) incubating the denatured nucleic acid sample with an exonuclease in reaction conditions which include at least one metal ion co-factor at a concentration wherein the exonuclease activity of said exonuclease is substantially suppressed and the endonuclease activity substantially retained or enhanced; and

iv) analyzing the products of the exonuclease activity of (iii) above.

According to a further aspect of the invention there is provided a method for the detection of sequence variation in nucleic acid target substrates comprising:

i) providing a nucleic acid target suspected of containing sequence variation relative to a reference control;

ii) providing conditions which enable said nucleic acid to form one or more secondary structures;

iii) providing an exonuclease wherein said exonuclease is provided with reaction conditions which include at least one metal ion cofactor at a concentration wherein the exonuclease activity is substantially suppressed and the endonuclease activity substantially retained or enhanced;

iv) creating a reaction mixture of (ii) and (iii) which generates multiple cleavage products;

v) separating said multiple cleavage products; and

vi) detecting the multiple cleavage products in (v).

According to a yet further aspect of the invention there is provided an assay for comparing a nucleic acid with a comparison nucleic acid sequence comprising:

i) incubating a denatured nucleic acid with at least two probes, one of which is labelled, both of which are adapted to bind to different parts of the nucleic acid;

ii) incubating the complex of nucleic acid and probes with an exonuclease in reaction conditions which include at least one metal ion co-factor at a concentration wherein the exonuclease activity of said exonuclease is substantially suppressed and the endonuclease activity substantially retained or enhanced; and

iii) comparing the products of the activity of the exonuclease of (ii) above with the products obtained by performing steps (i) and (ii) on the comparison nucleic acid.

According to a yet further aspect of the invention there is provided a method for performing a combined polymerase chain reaction (PCR) amplification and hybridization probing comprising the steps of:

i) contacting a target nucleic acid with PCR reagents, including at least two PCR primers, an exonuclease with both exonuclease and endonuclease activity under reaction conditions which include at least one metal ion co-factor at a concentration wherein the exonuclease activity is substantially suppressed and the endonuclease activity substantially retained or enhanced; the oligonucleotide probe comprising:

a) an oligonucleotide capable of hybridizing to a target nucleic acid;

b) a fluorescer molecule attached to the first end of the oligonucleotide;

c) a quencher molecule attached to a second end of the oligonucleotide such that the quencher molecule substantially quenches the fluorescer molecule whenever the oligonucleotide probe is in a single-stranded state and such that the fluorescer is substantially unquenched whenever the oligonucleotide probe is hybridized to the target nucleic acid;

d) a 5′ end which is rendered impervious to digestion by an exonuclease; and

e) a 3′ end which is rendered impervious to digestion by an exonuclease; and

ii) subjecting the target nucleic acid, the oligonucleotide probe, and the PCR reagents to thermal cycling, including a polymerization step, the thermal cycling being sufficient to amplify the target nucleic acid specified by the PCR reagents.

In a preferred method of the invention said exonuclease activity is a 5′-3′ exonuclease activity.

In a further preferred method of the invention said metal ion cofactor is not Mg²⁺.

In a yet further preferred method of the invention said metal ion cofactor is provided at a concentration of from between about 0.01 and 2.0 mM. Preferably said metal ion cofactor is provided at a concentration from between about 0.05 and 1.0 mM. More preferably still said metal ion cofactor is provided from between 0.1 and 0.5 mM. Preferably said metal ion cofactor is provided at about 0.1 mM.

Genotyping assays as herein disclosed typically use metal ion co-factors (e.g. Co⁺², Zn⁺²) since the enzymes used in said assays require metal ions for activity. We have discovered that the relative activities (i.e. exonuclease and endonuclease) of exonucleases can be altered by decreasing the concentration of metal ion cofactors by one or two orders of magnitude (e.g., from 10 mM used in conventional genotyping assays to 1.0-0.1 mM). In particular, this technique is particularly effective at suppressing exonuclease activity whilst retaining wild-type levels of endonuclease activity with low concentrations of transition metals.

In a preferred method of the invention said metal ion cofactor is selected from the transition metal group. Preferably said transition metal is selected from the group consisting of the following: Zn⁺², Co⁺², Ni⁺², or Cu⁺². Ni⁺² and Co⁺² are preferred, especially Co⁺².

In yet a still further preferred method of the invention said exonuclease is derived from DNA polymerase I. Preferably said exonuclease is derived from a bacterial DNA polymerase I. More preferably still said bacterial species is selected from; E.coli; Dienococcus radiodurans; Mycobacterium tuberculosis; Neisseria meningitdis; Mycoplasma spp.; Haemophilus spp.; Heliobacter spp.

In other preferred embodiments said bacterial species is a thermophilic bacterial species. More preferably still said thermophilic bacterial species is selected from; Thermus aquaticus; Thermus thermophilus; Thermosipho africanus; Thermotosa maritima. Alternatively said exonuclease is a 5′-3′ exonuclease of Aquifex aeolicus.

DNA polymerase I homologues (mesophilic or thermophilic bacterial species) with at least 25% homology to the first 250 amino acids of E. coli DNA polymerase I are the preferred DNA polymerase enzymes.

In a yet still further preferred method of the invention said exonuclease is phage exonuclease. More preferably still said phage exonuclease is a 5′-3′ exonuclease of T3 phage; T4 phage; T5 phage; T7 phage; or BF23 phage.

According to a yet further aspect of the invention there is provided a kit comprising: an exonuclease; optionally deoxynucleotide triphosphates; buffers which include metal ion cofactors selected from the group consisting of Zn⁺², Co⁺², Ni⁺², or Cu⁺²; standard DNA (undigested); standard DNA (digested); oligonucleotide primers; and optionally additional cofactors required by the exonuclease.

An embodiment of the invention will now be described, by way of example only, and with reference to the following figures.

### BRIEF DESCRIPTION OF THE FIGURES

FIG. 1 represents a diagrammatic representation of some of the 5′→3′ exonuclease and endonuclease nucleic acid substrates.

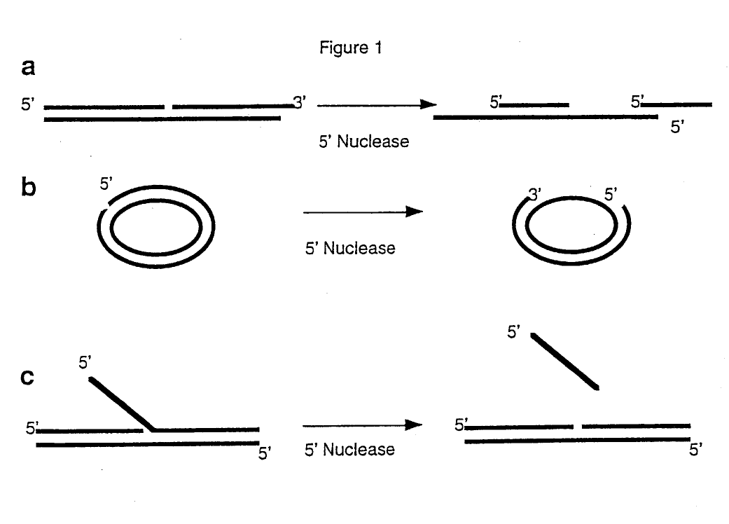

FIG. 2 is a comparison of wild-type T5 exonuclease activity using reaction conditions which include metal ions as co-factors at varying concentrations.

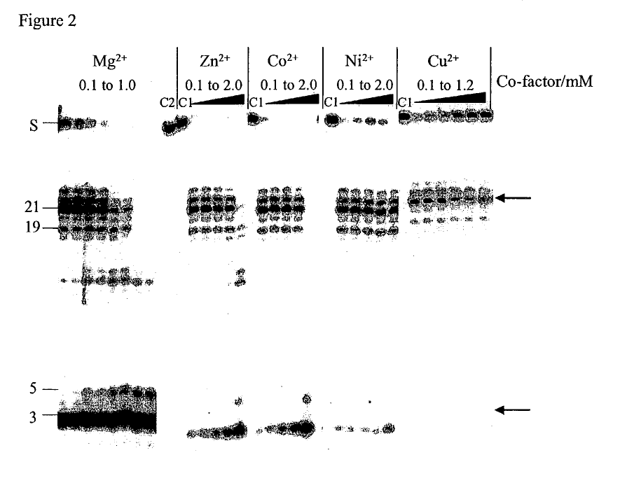

### EXAMPLE

Materials and Methods

Protein Expression and Purification of T5 D15 Exonuclease

The wild-type T5 exonuclease was subcloned into the expression vector pJONEX4 as described (1, 4). The wild-type protein was purified as described (1). Protein concentration was determined using the Bradford assay (5). The proteins were purified by ion exchange chromatography and eluted using a linear salt gradient between 0 and 1000 mM NaCl Fractions containing purified enzyme (as judged by analytical SDS-PAGE) were pooled, concentrated by ultrafiltration and stored in 50% glycerol (buffer A) at −20° C.

Nuclease Activity Assays

The release of acid-soluble nucleotides from high molecular weight DNA (herring sperm Type XIV) was determined with a standard UV spectrophotometric assay (1), except that the assay contained DNA at 670 μg/ml in 600 μl 25 mM potassium glycinate, pH 9.3, 10 mM MgCl₂ and 1 μg protein. Curves were plotted from the data obtained and estimates of the initial velocity were calculated.

Oligonucleotide Purification and Labelling

Pseudo-Y substrate was prepared by annealing 5′-³²P-end labeled FLAP oligonucleotide:

d(GATGTCAAGCAGTCCTAACTTTGAGGCAGAGTCC) (SEQ ID NO:1)

with BRIDGE oligonucleotide:

d(GGACTCTGCCTCAAGACGGTAGTCAACGTG) (SEQ ID NO:2)

essentially as described (7, 8). The FLAP oligonucleotide was end-labeled with [³²P]ATP under standard conditions and purified from a 7M urea-15% acrylamide gel essentially as described (9).

Standard Structure-specific DNA Cleavage

Structure specific cleavage of pseudo-Y and single-stranded DNA substrates by wild-type T5 exonuclease (final concentration 600 nM) were examined essentially as described (8) except the substrates were diluted in 10 mM Tris-HCl pH 8.0, 50 mM KCl, 0.5 mg/ml acetylated BSA, 10 mM MgCl₂ and incubated for various times. The enzyme was diluted in 10 mM Tris-HCl pH 8.0, 1 mM EDTA, 10% glycerol, 1 mM DTT and 1 mg/ml acetylated BSA. A final concentration of EDTA of 0.2 mM was used. Reactions were performed at room temperature.

Structure-specific Digest of Pseudo-Y Substrate by Wild-type T5 exonuclease Using Low Concentration of Metal Co-factors to Suppress Exonuclease Activity

T5 exonuclease at 650 nM was incubated with the substrate in the presence of various concentrations of magnesium chloride (Mg²⁺). zinc acetate (Zn²⁺), cobalt chloride (Co²⁺), nickel chloride (Ni²⁺), or copper sulphate (Cu²⁺). Reactions with Mg²⁺ were time-course of 30 minutes and 60 minutes at concentration of 0.1 mM (first 2 lanes), 0.2 mM (2^{nd} pair of lanes), 0.5 mM (3^{rd} pair of lanes), and 1.0 mM (4^{th} pair of lanes) respectively, because either higher concentration of Mg²⁺ or longer time scale will cause all the substrate to be degraded down to 3mer or 5mer products. All the other reactions were over night incubation at room temperature to avoid evaporation. Concentrations of Zn²⁺, Co²⁺ and Ni²⁺, were 0.1 mM, 0.2 mM, 0.5 mM, 1.0 mM, and 2.0 mM respectively. Concentrations for Cu²⁺ were 0.1 mM, 0.2 mM, 0.5 mM, 0.8 mM, 1.0 mM and 1.2 mM. Lane C2: control lane as enzyme was omitted from the reaction (Mg as co-factor); Lane C1: control lane as co-factors were omitted from the reaction. Upper arrow shows major endonucleolytic products, lower arrow, exonucleolytic products. Note, even at the lowest concentrations of Mg²⁺ used the exonucleolytic products predominate in the case of Mg²⁺ (Mn gives same profile as Mg).

### REFERENCES

1. Sayers, J R. and Eckstein, F. (1990) J. Biol. Chem., 265, 18311-18317.

2. Sayers, J R. and Krekel, C. and Eckstein, F. (1992) Biotechniques, 13, 592-596.

3. Sanger, F. Nicklen, S. and Coulsen, A R. (1977) Proc. Natl. Acad. Sci. USA, 80, 5463-5467.

4. Sayers, J R. and Eckstein, F. (1991) Nucl. Acids. Res., 19, 4127-4132.

5. Bradford, M M. (1976) Anal. Biochem., 72, 248-254.

6. Rosenthal, A L. and Kacks, S A. (1977) Anal. Biochem., 80, 76-90.

7. Harrington, J J. and Lieber, M R. (1994) EMBO J., 13, 1235-1246.

8. Garforth, S J. and Sayers, J R. (1997) Nucl. Acids. Res., 25, 3801-3807.

9. Sambrook, J., Fritsch, E F. and Maniatis, T. (1989) Molecular Cloning: A Laboratory Manual, 2nd Edn. Cold Spring Harbor Laboratory Press, Cold Spring Harbor, N.Y.

10. Pickering, T J, Garforth, S J, Sayers, J R & Grasby, J A. (1999) J. Biol. Chem. 274:17711-17717.

11. Carey, J. (1991) Methods Enzymol., 208, 103-117.

12. Engelke, D R, Krikos, A, Bruck, M E, Ginsburg D. (1900) Purification of Thermus aquaticus DNA-polymerase expressed in Escherichia-coli. Analytical Biochemistry 191:(2) 396-400.

13. Yuckenberg, P D, Witney, F, Geisselsoder, J and McClary, J. (1991) Site directed in vitro mutagenesis using uracil-containing DNA and phagemid vectors in “Directed mutagenesis; a practical approach” M J McPherson (ed), IRL Press at Oxford University Press, Oxford.

14. di Giovine, F S., Takhsh, E., Blakemore, A I F., and Duff, G W. (1992) Single base change at −511 in the human interleukin 1β-gene (IL1B). Hum. Mol. Genet., 1, 450.

15. Brow, A. A. D., Oldenburg, M., Lyamichev, V., Heisler, L., Grotelueschen, J., Lyamichev, N., Kozyavkin, S., Fors, L., Dahlberg, J., Smith, L., and Olive, D.M. (1996) Mutation Detection by Cleavase Fragment Length Polymorphism Analysis. FOCUS, 18 (1), 2-5.

The present invention is not limited in scope by the examples provided, since the examples are intended as illustrations of various aspects of the invention and other functionally equivalent embodiments are within the scope of the invention. Various modifications of the invention in addition to those shown are described herein will become apparent to those skilled in the art for the foregoing description and fall within the scope of the appended claims. The advantages and objects of the invention are not necessarily encompassed by each embodiment of the invention.

All references, patents, and patent publications that are recited in this application are incorporated in their entirety herein by reference.

1. A method for exposing a nucleic acid to the endonuclease activity of an exonuclease having both exonuclease and endonuclease activity, comprising forming a preparation containing the nucleic acid, the exonuclease and a metal ion co-factor at a concentration sufficient to suppress the exonuclease activity without suppressing its endonuclease activity to such an extent.

2. The method of claim 1, wherein the method is for comparing a nucleic acid with a comparison nucleic acid sequence comprising the steps of:
(i) providing a sample of nucleic acid to be assayed;
(ii) providing conditions for the denaturation of the nucleic acid;
(iii) incubating the denatured nucleic acid sample with an exonuclease in reaction conditions which include at least one metal ion co-factor at a concentration wherein the exonuclease activity of said exonuclease is substantially suppressed and the endonuclease activity substantially retained or enhanced; and
(iv) analyzing the products of the exonuclease activity of (iii) above.

3. The method of claim 1, wherein the method is for the detection of sequence variation in nucleic acid target substrates comprising the steps of:
(i) providing a nucleic acid target suspected of containing sequence variation relative to a reference control;
(ii) providing conditions which enable said nucleic acid to form one or more secondary structures;
(iii) providing an exonuclease wherein said exonuclease is provided with reaction conditions which include at least one metal ion cofactor at a concentration wherein the exonuclease activity is substantially suppressed and the endonuclease activity substantially retained or enhanced;
(iv) creating a reaction mixture of (ii) and (iii) which generates multiple cleavage products;
(v) separating said multiple cleavage products; and
(vi) detecting the multiple cleavage products in (v).

4. The method of claim 1, wherein the method is for comparing a nucleic acid with a comparison nucleic acid sequence comprising the steps of:
(i) incubating a denatured nucleic acid with at least two probes, one of which is labelled, both of which are adapted to bind to different parts of the nucleic acid;
(ii) incubating the complex of nucleic acid and probes with an exonuclease in reaction conditions which include at least one metal ion co-factor at a concentration wherein the exonuclease activity of said exonuclease is substantially suppressed and the endonuclease activity substantially retained or enhanced; and
(iii) comparing the products of the activity of the exonuclease of (ii) above with the products obtained by performing steps (i) and (ii) on the comparison nucleic acid.

5. The method of claim 2, wherein the method is for comparing a nucleic acid with a comparison nucleic acid sequence comprising the steps of:
(i) incubating a denatured nucleic acid with at least two probes, one of which is labelled, both of which are adapted to bind to different parts of the nucleic acid;
(ii) incubating the complex of nucleic acid and probes with an exonuclease in reaction conditions which include at least one metal ion co-factor at a concentration wherein the exonuclease activity of said exonuclease is substantially suppressed and the endonuclease activity substantially retained or enhanced; and
(iii) comparing the products of the activity of the exonuclease of (ii) above with the products obtained by performing steps (i) and (ii) on the comparison nucleic acid.

6. The method of claim 1, wherein the method is for performing a combined polymerase chain reaction (PCR) amplification and hybridization probing comprising the steps of:
(i) contacting a target nucleic acid with PCR reagents, including at least two PCR primers, an exonuclease with both exonuclease and endonuclease activity under reaction conditions which include at least one metal ion co-factor at a concentration wherein the exonuclease activity is substantially suppressed and the endonuclease activity substantially retained or enhanced; the oligonucleotide probe comprising:
(a) an oligonucleotide capable of hybridizing to a target nucleic acid;
(b) a fluorescer molecule attached to the first end of the oligonucleotide;
(c) a quencher molecule attached to a second end of the oligonucleotide such that the quencher molecule substantially quenches the fluorescer molecule whenever the oligonucleotide probe is in a single-stranded state and such that the fluorescer is substantially unquenched whenever the oligonucleotide probe is hybridized to the target nucleic acid;
(d) a 5′ end which is rendered impervious to digestion by an exonuclease; and
(e) a 3′ end which is rendered impervious to digestion by an exonuclease; and
(ii) subjecting the target nucleic acid, the oligonucleotide probe, and the PCR reagents to thermal cycling, including a polymerisation step, the thermal cycling being sufficient to amplify the target nucleic acid specified by the PCR reagents.

7. The method of claim 1, wherein the exonuclease activity is a 5′-3′ exonuclease activity.

8. The method of claim 1, wherein the metal ion cofactor is not Mg²⁺.

9. The method of claim 1, wherein the metal ion cofactor is provided at a concentration of from between about 0.01 and about 2.0 mM.

10. The method of claim 9, wherein the metal ion cofactor is provided at a concentration from between about 0.05 and about 1.0 mM.

11. The method of claim 10, wherein the metal ion cofactor is provided from between about 0.1 and about 0.5 mM.

12. The method of claim 11, wherein the metal ion cofactor is provided at about 0.1 mM.

13. The method of claim 1, wherein the metal ion cofactor is selected from the transition metal group.

14. The method of claim 13, wherein the transition metal is selected from the group consisting of: Zn⁺², Co⁺², Ni⁺² and Cu⁺².

15. The method of claim 14, wherein the transition metal is Ni⁺² or Co⁺².

16. The method of claim 15, wherein the transition metal is Co⁺².

17. The method of claim 1, wherein the exonuclease is derived from DNA polymerase I.

18. The method of claim 17, wherein the exonuclease is derived from a bacterial DNA polymerase I.

19. The method of claim 18, wherein the bacterial DNA polymerase is derived from a bacterial species selected from the group consisting of: E. coli; Dienococcus radiodurans; Mycobacterium tuberculosis; Neisseria meningitdis; Mycoplasma spp.; Haemophilus spp.; and Heliobacter spp.

20. The method of claim 18, wherein the DNA polymerase I is a thermophilic DNA polymerase.

21. The method of claim 20, wherein the thermophilic DNA polymerase I is derived from a bacterial species selected from the group consisting of: Thermus aquaticus; Thermus thermophilus; Thermosipho africanus; Thermotosa maritima; and Aquifex aeolicus.

22. The method of claim 17, wherein the DNA polymerase I is a DNA polymerase with at least 25% homology to the first 250 amino acids of E.coli DNA polymerase I.

23. The method of claim 1, wherein the exonuclease is phage exonuclease.

24. The method of claim 23, wherein the phage exonuclease is a 5′→3′ exonuclease of a phage selected from the group consisting of: T3 phage; T4 phage; T5 phage; T7 phage; and BF23 phage.

25. A kit comprising: an exonuclease; deoxynucleotide triphosphates; buffers which include metal ion cofactors selected from the group consisting of Zn⁺², Co⁺², Ni⁺², or Cu⁺²; standard DNA (undigested); standard DNA (digested); oligonucleotide primers; and optionally additional cofactors required by the exonuclease.


SEQUENCE LISTING (2 sequences)

SEQ ID NO: 1 - DNA, 34 bp, Artificial Sequence
  Description of Artificial Sequence synthetic oligonucleotide
gatgtcaagcagtcctaactttgaggcagagtcc

SEQ ID NO: 2 - DNA, 30 bp, Artificial Sequence
  Description of Artificial Sequence synthetic oligonucleotide
ggactctgcctcaagacggtagtcaacgtg